(19) United States
(12) Patent Application Publication
(10) Pub. No.: US 2007/0207091 A1
(43) Pub. Date: Sep. 6, 2007

(54) Nebulizer Formulation

(76) Inventors: Ian Gardner Cameron McAFFER, Biggin Hill (GB); Peter Ernest Tasko, Hitchin (GB)

Correspondence Address: STERNE, KESSLER, GOLDSTEIN & FOX P.L.L.C., 1100 NEW YORK AVENUE, N.W., WASHINGTON, DC, 20005, US

(21) Appl. No.: 11/680,696

(22) Filed: Mar. 1, 2007

(30) Foreign Application Priority Data
Mar. 1, 2006 (GB) 0604141.2

(51) Int. Cl. A61K 31/573; A61K 9/12; A61M 11/00

(52) U.S. Cl. 424/45; 514/171; 128/200.23

(57) ABSTRACT

A sterile nebulizer formulation contains formoterol and budesonide in about 2 ml or less of saline and is for treatment of COPD and asthma and other airways diseases and disorders.

### BACKGROUND OF THE INVENTION

The present invention relates to a nebulizer formulation, in particular a nebulizer formulation, a method of manufacturing the formulation and a method of treatment for diseases such as Chronic Obstructive Pulmonary Disease (COPD) and asthma using the formulation.

Nebulizers provide a means of administering drugs to the airways of a patient whilst the patient breathes at an approximately normal rate. They are particularly suitable for patients who are unable, whether due to age or injury or otherwise, to inhale at the much higher rates required for administration of drugs via metered dose inhalers or dry powder inhalers and for patients who cannot for whatever reason coordinate the activation of the metered dose inhaler with their inhalation of breath. The nebulizer apparatus creates a vapour containing drug particles and the patient breathes the vapour via a mouthpiece or mask attached to the nebulizer. Typically, nebulizers are used to deliver drugs for the treatment of airways disorders such as asthma and COPD.

According to the U.S. Centers for Disease Control and Prevention, COPD is currently the fourth leading cause of death in the U.S. (behind heart disease, cancer and stroke), claiming the lives of in excess of 100,000 Americans annually. An estimated 16 million Americans have been diagnosed with some form of COPD, and as many as 16 million others have the condition but have not yet been diagnosed. COPD is hence regarded as a major and growing health care threat in the U.S. and throughout the rest of the world.

A known formulation for treatment of COPD comprises albuterol (also known as salbutamol) and ipratropium in an ampoule containing 3.0 ml of solution, and is described in WO 03/037159 and the equivalent U.S. Pat. No. 6,632,842. In use, the contents of the ampoule are poured into the chamber of the nebulizer and the patient then breathes the vapour generated until the ampoule contents are used. Treatment is typically required up to 4 times per day, at regular intervals.

Low patient compliance is a known problem with nebulized drugs generally, as the period of nebulizing required to administer a dose is long, typically tens of minutes, perhaps half-an-hour for a typical dose. Children and adults can become bored during this period. Patients who stop nebulizing prematurely do not receive a full dose. This can in turn lead to further reduced patient compliance as the inadequate dose fails to provide adequate therapy, discouraging further use.

Treatment of COPD by formoterol and budesonide dry powder inhalation is known (US application 2005/0042175) but powdered formulations are more difficult to administer than liquid formulations that can be administered by nebulization.

US application 2003/0055026 discloses a method of COPD treatment by nebulization of a formoterol and budesonide composition and refers to filter-sterilization, but this method is not in fact suitable for budesonide sterilization, as shown in WO 99/25359.

It is an object of the present invention to provide formulations, a method of manufacturing formulations and the use of these formulations which overcome or at least ameliorate one or more of the above-identified problems.

### SUMMARY OF THE INVENTION

The present invention provides novel, sterilized nebulizer formulations, suitable for treatment of COPD, asthma and other conditions associated with reversible obstruction of the airways. The formulations can be utilized in a variety of known nebulizer apparatus, with potential reduced wastage of ingredients and/or an anticipated increase in patient compliance. The invention provides a method of treatment of COPD, asthma and other conditions associated with reversible obstruction of the airways comprising administering, via a nebulizer, a sterilized formulation containing both formoterol and budesonide in a pharmaceutically acceptable carrier.

The invention further provides a method of manufacturing sterile nebulizer formulations, by combining formoterol and budesonide under nitrogen gas before filling into ampoules.

The present invention provides a method of treatment of COPD, asthma and other conditions associated with reversible obstruction of the airways, comprising providing a nebulizer and an ampoule containing not more than 2.2 ml of a formulation comprising formoterol and budesonide in a pharmaceutically acceptable carrier, and administering the formulation using the nebulizer.

A filled ampoule of the invention contains a sterile formulation of formoterol and budesonide.

Also provided by the invention is a method that is expected to increase patient compliance in use of a nebulizer formulation, comprising providing a nebulizer, and an ampoule containing said formulation, wherein the formulation comprises formoterol and budesonide in a pharmaceutically acceptable carrier and wherein the ampoule contains not more than 2.2 ml and not less than 0.3 ml of said formulation, and administering the formulation using the nebulizer.

A kit of the invention comprises a sterile formulation of the invention with instructions on how to use it.

### DETAILED DESCRIPTION OF THE PREFERRED EMBODIMENTS

The invention enables use of nebulizer formulations without the lower limit on volume typically associated with the art. The formoterol, a beta agonist, is formulated with and administered with budesonide, a steroid, in a sterile formulation.

Reference to these active agents herein is intended to refer also to pharmaceutically acceptable derivatives thereof, such as but not limited to salts, esters, enol ethers, enol esters, acids, bases, solvates, hydrates or prodrugs thereof. Budesonide is a corticosteroid that reduces the frequency and severity of COPD/asthma exacerbations, and which may synergize with the bronchodilator effect of formoterol. Reference to budesonide thus includes, but is not limited to, any form of budesonide which inhibits disease exacerbations in patients suffering from COPD, including, but not limited to, all automatic forms, enantiomer forms, stereoisomer, anhydrides, acid addition salts, base salts, solvates, analogues and derivatives of budesonide.

A method of treatment of COPD or asthma using the teachings of the invention comprises administering to a human patient, via a nebulizer, a sterile formulation containing effective amounts of both formoterol and budesonide in a pharmaceutically acceptable carrier, and it is expected, using this ampoule formulation, to achieve improved acceptance of the medicine and better patient compliance.

The compositions provided herein are used for treating, preventing, or ameliorating one or more symptoms of a bronchoconstrictive disorder or disease in a human subject. In one embodiment, the method includes nebulizer administration to a subject of an effective amount of a sterile composition containing formoterol and budesonide, whereby the disease or disorder is treated or prevented.

The invention relates in particular to formulations for treatment of COPD and asthma, including, but not limited to, chronic bronchitis, emphysema, and associated cor pulmonale (heart disease secondary to disease of the lungs and respiratory system) with pulmonary hypertension, right ventricular hypertrophy and right heart failure, and also bronchial asthma, allergic asthma and intrinsic asthma, e.g., late asthma and airway hyper-responsiveness.

The formulations of the present invention are designed for administration by nebulizer. A nebulized solution is one dispersed in air to form an aerosol, and a nebulizer generates very fine liquid droplets suitable for inhalation into the lung. Nebulizers typically use compressed air, ultrasonic waves, or a vibrating mesh to create a mist of the droplets and may also have a baffle to remove larger droplets from the mist by impaction. A variety of nebulizers are available for this purpose, such as ultrasonic nebulizers, jet nebulizers and breath-actuated nebulizers. In use, mouthpieces or masks are typically attached to a patient to aid delivery of the nebulized solution.

In preferred embodiments of the invention, formulations are for delivery with and patients are treated using a high efficiency nebulizer, in particular one that can deliver at least 15%, preferably at least 25%, more preferably at least 35% of the drug substance to the patient's lungs.

In specific embodiments of the invention, formulations are delivered using a high efficiency jet nebulizer, a high efficiency ultrasonic nebulizer or a high efficiency vibrating mesh nebulizer, use of these devices enabling and/or enhancing the use of the reduced volume formulations of the invention. Jet nebulizers are particularly preferred, and one example is the PARI LC Plus (registered trade mark, Pari GmbH, Germany) nebulizer.

The invention is of particular application to COPD. Specifically, a method of treatment of COPD according to the present invention hence comprises:
(1) providing:
   a) a nebulizer, and
   b) an ampoule containing not more than 2.2 ml of a sterile formulation comprising formoterol and budesonide in a pharmaceutically acceptable carrier, and
(2) administering the formulation using the nebulizer.

Preferably, the invention provides and uses ampoules which contain not less than 0.5 ml of formulation, more preferably about 1.0 to 2 ml of said formulation, and very preferably about 1.5 ml to 2 ml of said formulation. These reduced volumes can lead to significant reductions in treatment times, with the expected advantages explained.

Formulations and compositions of the invention generally comprise a pharmaceutically acceptable carrier. The carrier is preferably a liquid carrier. Further, the carrier preferably comprises water and may comprise other components.

A filled ampoule of the invention contains a formulation of formoterol and budesonide. This is generally in a pharmaceutically acceptable carrier and buffered for human use to a pH of about 3.5-5.5. The formulations of the examples are buffered to about pH 4. The formulations are optionally free of preservative, which is an advantage as some preservatives can be associated with bronchoconstrictor effects—the opposite effect to that required by the formulation. Water is used to provide the carrier, and water for injection is preferred due to its purity.

One or more tonicity adjusting agents may be added to provide the desired ionic strength. Tonicity adjusting agents for use herein include those which display no or only negligible pharmacological activity after administration. Both inorganic and organic tonicity adjusting agents may be used. Compositions of the invention can also include excipients and/or additives. Examples of these are surfactants, stabilizers, complexing agents, antioxidants, or preservatives which prolong the duration of use of the finished pharmaceutical formulation, flavorings, vitamins, or other additives known in the art. Complexing agents include, but are not limited to, ethylenediaminetetraacetic acid (EDTA) or a salt thereof, such as the disodium salt, citric acid, nitrilotriacetic acid and the salts thereof. In one embodiment, the complexing agent is EDTA. Preservatives include, but are not limited to, those that protect the solution from contamination with pathogenic particles, including benzalkonium chloride or benzoic acid, or benzoates such as sodium benzoate. Antioxidants include, but are not limited to, vitamins, provitamins, ascorbic acid, vitamin E or salts or esters thereof.

Formulations as described in this invention can be readily prepared by a person of skill in the art. In one method, a solution of NaCl is prepared with concentration approximately 9 g/l. To this are added budesonide to a concentration as desired, but typically about 0.25 mg/ml and formoterol, again to the concentration desired but typically about 0.625 mg/ml. HCl is then added to give a final pH of about 4.0. This formulation can be filled into ampoules using blow fill seal technology (described in more detail below) to yield ampoules with the required extractable volume of formulation.

Reference to an ampoule with a specified volume and to the extractable volume of an ampoule refer to the volume of solution that can be extracted from the ampoule in normal use, e.g. by breaking it open and pouring out the contents without actively flushing the ampoule or carrying out scientific extraction methods. There is in addition some tolerance in the volumes recited, as filling machines vary in their accuracy. By way of illustration, “an ampoule containing 2 ml of solution” and a “2 ml ampoule”, say, both refer to an ampoule which contains about 2.1 to about 2.2, generally about 2.15, ml of solution and which when opened and poured into the nebulizer results in approximately 2 ml of solution being transferred into the nebulizer. Hence, the volumes recited refer to the amount of solution that can be readily extracted from the ampoule rather than the amount the ampoule is filled with.

Preferably, ampoules of the invention have reduced volume, containing 2.2 ml or less of said formulation, preferably 2.0 ml or less of said formulation or about 1.0 to 2 ml of said formulation. Specific embodiments of the invention, set out in detail below, provide ampoules of about 2 ml. Other suitable ampoule volumes are about 1.5 ml, about 1.0 ml and about 0.5 ml.

By using the formulations of the invention, it is possible to deliver a sufficient dose of formoterol and budesonide in a shorter period of time than is necessary for known combination formulations. Hence, the invention provides in another aspect a method of increasing patient compliance in use of a nebulizer formulation, comprising
(1) providing:
   a) a nebulizer, and
   b) an ampoule containing said formulation, wherein the formulation comprises formoterol and budesonide in a pharmaceutically acceptable carrier and wherein the ampoule contains not more than 2.2 ml and not less than 0.3 ml of said formulation, and
(2) administering the formulation using the nebulizer.

The volume of the ampoule can be reduced following the teachings of the invention. However, there is a practical limit to the concentration of the contents of ampoules of the invention in as much as very small amounts of highly concentrated liquids are easily spilled and are not so easy to dispense accurately. In preferred methods and formulations, the ampoules contain not more than 2 ml and not less than 0.5 ml of said formulation.

The formulation used in the method typically contains from 3 to 40 mcg of formoterol, preferably from 10 to 15 mcg of formoterol. The formulation also typically contains from 0.25 to 1.0 mg of budesonide, preferably from 0.40 to 0.70 mg of budesonide. These formulations preferably have volumes of about 2.0 ml, about 1.5 ml, about 1.0 ml or about 0.5 ml.

Pharmaceutical compositions containing formoterol and a budesonide for administration via nebulization are hence provided. The compositions are sterilized and filled in ampoules or vials, including unit dose vials, providing sterile unit dose formulations for use in a nebulizer. By sterile, it is meant that the resultant pharmaceutical composition meets the requirements of sterility enforced by medicine regulatory authorities, such as the FDA in the US or the MCA in the UK. Tests are included in current versions of the compendia, such as the US Pharmacopoeia and the British Pharmacopoeia.

Formoterol is preferably filter sterilized. Budesonide can be sterilized by rapid heat treatment or solvent treatment, as described in WO 02/41925 and WO 03/070285, respectively. Formoterol may be prepared according to the method disclosed in U.S. Pat. No. 3,994,974, and the individual enantiomers of formoterol may be prepared by the method disclosed in U.S. Pat. No. 6,040,344. Budesonide may be synthesized by the procedure disclosed in U.S. Pat. No. 3,929,768.

In a further embodiment, the present invention provides a container containing a vial, comprising a single unit dose of a therapeutically effective amount of formoterol and budesonide in a sterile solution, or a plurality of such vials, for use in a nebulizer.

The extractable volume of each unit dose of a specific embodiment of the invention comprises about 12 mcg of formoterol (or equivalent amount of a derivative thereof) and about 0.5 mg budesonide (or equivalent amount of a derivative thereof) in a sterile, aqueous solution. The solution comprises sodium chloride at about 9 mg/ml to make the solution isotonic and hydrochloric acid to adjust pH of the solution to about 4.0. It is optional to include a chelating agent, such as EDTA. The volume is preferably about 2.0 ml or about 1.5 ml.

The invention additionally provides kits for use in treatment of the diseases described herein. The kits comprise:
(1) a container, containing a single, sterile unit dose of a therapeutically effective amount of formoterol and budesonide; and
(2) instructions on how the dose is to be used in a nebulizer.

The single unit dose is suitably as described elsewhere herein in relation to formulations of the invention. The instructions instruct the patient as to how the dose should be used in conjunction with a nebulizer, such as how to open it and transfer its contents into the nebulizer, how to operate the nebulizer and for how long nebulizing should be continued to complete administration of the dose.

Kits of the invention can contain a plurality of single unit doses for use in a nebulizer. One kit comprises at least 120 or at least 125 single unit doses, being designed to provide one month's worth of doses to be taken 4 times per day. Another kit comprises at least 25 or at least 28 single unit doses, designed for a week's supply at 4 per day. Other kits may usefully contain 30 or 60 single unit doses.

For embodiments of the invention in which the extractable volume is 2.2 ml or less, especially where the volume is 2.0 ml or 1.5 ml or lower, the instructions may explain that the present formulation can be administered in less time than a previously known formulation, such as a known 3 ml formulation, hence reinforcing this advantage of the invention and improving the prospects for increased patient compliance. Preferably, the instructions explain that the patient should continue administering the dose until the complete dose has been administered.

In embodiments of the invention, sterile formoterol and sterile budesonide are combined under nitrogen. In a specific embodiment, sterile budesonide is compounded in an isolated, closed system with absolute microbiological control. Sterilized budesonide is used in a concentrated form to make a bulk solution by suspension in a surfactant. The budesonide suspension is combined with filter-sterilized formoterol in a pre-sterilized filling tank containing nitrogen gas under positive pressure, before filling into ampoules.

Formoterol is sensitive to degradation, and it is an anticipated advantage of the present invention that greater formulation stability is achieved by combining formoterol with budesonide under nitrogen.

Formulations of the invention are suitable for filling into ampoules using, “blow fill seal” (BFS) methods. The principle is that a plastic parison is extruded from polymer, formed into a container, filled and sealed in a single aseptic operation. BFS is now the preferred method for aseptic manufacture of ampoules due to the flexibility in container design, overall product quality, product output and low operational costs. Fill accuracies of better than ±5% have been demonstrated for container volumes as small as 0.5 ml and hence BFS is suitable for manufacture of ampoules according to the invention.

One BFS operation includes the multi-step process of blow moulding, aseptic filling and hermetic sealing of liquid products with fill volumes ranging from 0.1 ml to 1,000 ml, though for ampoules volumes in the range 0.5 ml to 5 ml are more common. A variety of polymers may be used in the process; low and high-density polyethylene and polypropylene are the, most popular.

Furthermore, the BFS process flow is normally impacted by only two raw materials—product and polymer—that are each processed inline, thereby making the process amenable to large uninterrupted batch sizes, some in excess of 500,000 or 1,000,000 units, and fill durations of up to 120 hours.

In a typical operation, to form the container, thermoplastic is continuously extruded in a tubular shape. When the tube reaches the proper length, the mould closes and the parison is cut. The bottom of the parison is pinched, closed and the top is held in place with a set of holding jaws. The mould is then transferred to a position under the filling station. To fill the container, the nozzle assembly lowers into the parison until the nozzles form a seal with the neck of the mould. Container formation is completed by applying vacuum on the mould side of the container and by blowing sterile filtered air into the interior of the container. The fill system delivers a precise dosage of product into the container. The nozzles retract into their original position. Lastly, to seal the container, following completion of the filling process, the top of the container remains semi-molten. Separate seal moulds close to form the top and hermetically seal the container. The moulds open and the container is then conveyed out of the machine. The whole of the above process is operated in pharmaceutical aseptic processing conditions.

BFS machines are commercially available from a number of suppliers, including Weiler Engineering, Inc (US) and Rommelag USA Inc (US).

The invention is now illustrated by the following non-limiting example.

### EXAMPLE

### Manufacture of Sterile Formoterol-Budesonide Nebulizer Formulation

A bulk nebulizer formulation is prepared in a pharmaceutically clean formulation tank that is 90% filled with low bioburden water for injection (WFI), and excipients and formoterol added. The contents are mixed thoroughly to ensure solubilization.

The solution is then passed through a 0.2 μm bacterial retaining filter and transferred to a pre-sterilized filling tank containing sterile nitrogen gas that has been passed through a 0.2 μm filter. A positive nitrogen pressure is maintained within the filling tank throughout the filling process.

A concentrate of sterile micronised budesonide is prepared within a pre-sterilized isolator and dispensed into a mixing tank containing Tween 80 that has been filter-sterilized through 0.2 μm pores. The tank contents are mixed, causing the budesonide; to be suspended in the Tween 80. The budesonide suspension is transferred via a sterile closed system to the filling tank, where it is mixed with the formoterol to form a formoterol and budesonide suspension. The filling tank is weighed and then filled to the final formulation weight with sterile WFI introduced via the isolator and sterile closed system.

The formoterol and budesonide formulation is then filled into sterile unit dose Blow Fill Seal ampoules via a BFS machine, whose product pathways and support systems have been previously sterilized to current industry standards. The resulting filled ampoules contain a 2 ml dose of a sterile formulation of 12 mcg formoterol and 0.5 mg budesonide. The invention thus provides sterile nebulizer formulations of formoterol and budesonide.

1. A method of treatment of Chronic Obstructive Pulmonary Disorder (COPD) or asthma in a patient in need thereof, comprising:
(1) providing:
a) a nebulizer, and
b) an ampoule containing not more than 2.2 ml of a formulation comprising formoterol and budesonide in a pharmaceutically acceptable carrier, and
(2) administering the formulation using the nebuliser to said patient.

2. The method of claim 1, wherein the ampoule contains not less than 0.5 ml of said formulation.

3. The method of claim 1, wherein the ampoule contains about 1.0 to 2 ml of said formulation.

4. The method of claim 1, wherein the ampoule contains about 1.5 ml to 2 ml of said formulation.

5. The method of claim 1, wherein the formulation is sterile.

6. A method of treatment of COPD or asthma in a patient in need thereof, comprising administering to said patient, via a nebulizer, a formulation comprising formoterol and budesonide in a pharmaceutically acceptable carrier.

7. The method of claim 6, wherein the patient has asthma.

8. The method of claim 6, wherein the patient has COPD.

9. The method of claim 6, wherein the formulation is sterile

10. A filled ampoule containing a formulation comprising formoterol and budesonide, and a pharmaceutically acceptable carrier.

11. The filled ampoule of claim 10, containing 2.2 ml or less of said formulation.

12. The filled ampoule of claim 10, containing about 2.0 ml of said formulation.

13. The filled ampoule of claim 10, containing about 1.0 to 2 ml of said formulation.

14. The filled ampoule of claim 10, wherein the formulation is sterile.

15. A method for increasing patient compliance in use of a nebulizer formulation to treat COPD or asthma, the method comprising providing an ampoule containing said formulation, wherein said formulation comprises formoterol and budesonide in a pharmaceutically acceptable carrier and wherein the ampoule contains not more than 2.2 ml and not less than 0.3 ml of said formulation.

16. The method of claim 15, wherein the ampoule contains not more than 2 ml and not less than 0.5 ml of said formulation.

17. The method of claim 15, wherein the formulation is sterile.

18. The method of claim 15 for treatment of COPD.

19. The method of claim 15, for treatment of asthma.

20. A kit comprising:
(1) a container, containing a single, sterile unit dose of a therapeutically effective amount of formoterol and budesonide, and a pharmaceutically acceptable carrier; and
(2) instructions on how the dose is to be used in a nebulizer for treatment of COPD or asthma.

21. The kit of claim 20, wherein the single unit does is 2.2 ml or less in volume, and the instructions explain that the single unit dose can be administered in less time than a previously known formulation

22. The kit of claim 20, wherein the instructions explain that the patient should continue administering the dose until the complete dose has been administered.

23. The kit of claim 20, wherein the single unit dose is about 2.0 ml in volume.

24. The kit of claim 20, comprising instructions to deliver the dose using a high efficiency nebuliser.

25. The kit of claim 24 wherein the nebuliser is a jet nebuliser.

26. The kit of claim 20, comprising at least 25 of said single unit doses.

27. The kit of claim 26, comprising at least 120 of said single unit doses.

28. A method for manufacturing a sterile nebuliser formulation, comprising combining sterilized formoterol and sterilized budesonide under nitrogen gas

29. The method of claim 28, comprising:
(1) preparing a sterile solution of formoterol,
(2) preparing a sterile suspension of budesonide, and
(3) combining the solution and the suspension under nitrogen.

30. The method of claim 29, comprising preparing the sterile solution by passing formoterol solution through a filter into a sterile tank containing nitrogen.

31. The method of claim 29 or 30, comprising passing a surfactant through a filter into a sterile tank, passing sterile, micronised budesonide into the tank and mixing the surfactant and the budesonide.

32. The method of any of claims 28 to 31 comprising filling sterile ampoules with the formulation by a Blow Fill Seal method.
